(19) United States
(12) Patent Application Publication
(10) Pub. No.: US 2022/0054817 A1
(43) Pub. Date: Feb. 24, 2022

(54) COVER FOR USE WITH SHEATH HUB AND DILATOR HUB

(71) Applicant: Baylis Medical Company Inc., Montreal (CA)

(72) Inventors: Jennifer Chan, Richmond Hill (CA); Jan-Hung Chen, Georgetown (CA)

(21) Appl. No.: 17/397,179

(22) Filed: Aug. 9, 2021

Related U.S. Application Data
(60) Provisional application No. 63/067,065, filed on Aug. 18, 2020.

(51) Int. Cl. A61M 39/20; A61M 29/02; A61M 39/10

(52) U.S. Cl. CPC A61M 39/20; A61M 2039/0229; A61M 39/10; A61M 29/02

(57) ABSTRACT

A snap-fit cover is configured to snap fit to a dilator hub. The snap-fit cover is also configured to snap fit to the sheath-hub cap of the sheath hub.

### TECHNICAL FIELD

This document relates to the technical field of (and is not limited to) (A) a cover for use with a sheath hub and a dilator hub (and method therefor); and (B) a snap-fit cover for use with a sheath hub and a dilator hub (and method therefor).

### BACKGROUND

Known medical devices are configured to facilitate a medical procedure, and help healthcare providers diagnose and/or treat medical conditions of sick patients.

### SUMMARY

It will be appreciated that there exists a need to mitigate (at least in part) at least one problem associated with existing (known) sheath hubs and dilator hubs (also called the existing technology). After much study of, and experimentation with, the existing (known) sheath hubs and dilator hubs, an understanding (at least in part) of the problem and its solution have been identified (at least in part) and are articulated (at least in part) as follows:

Known sheath-hub caps and known dilator hubs may be connected together by a connection joint. The known dilator hub may have a dilator ring at the distal end of the dilator hub. The dilator ring is configured to engage a known sheath hub cap at its proximal, circular opening. It has been observed that the known sheath-hub cap is made of a material having a relatively stiffer attribute compared to the stiffness of the material of the known dilator hub, which leads to unwanted (inadvertent) damage to the known dilator hub (when installing the known sheath-hub cap to the known dilator hub). The known sheath-hub cap may lead to the application of unwanted large shear stress to the known dilator ring (during connection of the sheath-hub cap to the dilator ring of the known dilator hub), which may lead to unwanted damage to the dilator ring and/or generate undesirable particulates (released from the damaged dilator). Sometimes, physicians complain about the material separating from the dilator hub due to damage of the dilator ring, which generates or forms unwanted particulates.

To mitigate, at least in part, at least one problem associated with the existing technology, there is provided (in accordance with an aspect) an apparatus. The apparatus is for use with a dilator hub and a sheath-hub cap configured to be selectively installed to a sheath hub. The apparatus includes and is not limited to (comprises) a snap-fit cover configured to snap fit to the dilator hub. The snap-fit cover is also configured to snap fit to the sheath-hub cap of the sheath hub.

To mitigate, at least in part, at least one problem associated with the existing technology, there is provided (in accordance with an aspect) an apparatus. The apparatus is for use with a dilator hub and a sheath-hub cap configured to be selectively installed to a sheath hub having a sheath-hub portal. The apparatus includes and is not limited to (comprises) a snap-fit cover defining a cover entrance portal configured to receive, at least in part, the dilator hub. The snap-fit cover is configured to snap fit to a portion of the dilator hub. The snap-fit cover is also configured to snap fit to the sheath-hub cap configured to be selectively installed after the sheath-hub cap has been installed to the sheath-hub portal of the sheath hub.

To mitigate, at least in part, at least one problem associated with the existing technology, there is provided (in accordance with an aspect) a method. The method is for connecting a sheath-hub cap of a sheath hub with a dilator hub. The method includes and is not limited to (comprises) snap fitting a snap-fit cover to the dilator hub. The method also includes snap fitting the snap-fit cover to the sheath-hub cap. It will be appreciated that, in accordance with a preferred embodiment, the snap-fit cover is snap fitted (connected) to the hub cap, and then the dilator hub is snap fitted (connected) to the snap-fit cover.

Other aspects are identified in the claims. Other aspects and features of the non-limiting embodiments may now become apparent to those skilled in the art upon review of the following detailed description of the non-limiting embodiments with the accompanying drawings. This Summary is provided to introduce concepts in simplified form that are further described below in the Detailed Description. This Summary is not intended to identify potentially key features or possible essential features of the disclosed subject matter, and is not intended to describe each disclosed embodiment or every implementation of the disclosed subject matter. Many other novel advantages, features, and relationships will become apparent as this description proceeds. The figures and the description that follow more particularly exemplify illustrative embodiments.

### BRIEF DESCRIPTION OF THE DRAWINGS

The non-limiting embodiments may be more fully appreciated by reference to the following detailed description of the non-limiting embodiments when taken in conjunction with the accompanying drawings, in which:

FIG. 1, FIG. 2 and FIG. 3 depict a perspective view (FIG. 1), a side view (FIG. 2) and a cross-sectional view (FIG. 3) of embodiments (implementations) of a snap-fit cover; and

FIG. 4 and FIG. 5 depict embodiments of a perspective view of the sheath hub cap and sheath hub (FIG. 4) and a cross-sectional view of the sheath hub cap (FIG. 5) for use with the snap-fit cover of FIG. 1; and

FIG. 6 and FIG. 7 depict side views of embodiments (implementations) of a dilator hub for use with the snap-fit cover of FIG. 1; and

FIG. 8 to FIG. 13 depict side views of embodiments (implementations) of the snap-fit cover of FIG. 1.

The drawings are not necessarily to scale and may be illustrated by phantom lines, diagrammatic representations and fragmentary views. In certain instances, details unnecessary for an understanding of the embodiments (and/or details that render other details difficult to perceive) may have been omitted. Corresponding reference characters indicate corresponding components throughout the several figures of the drawings. Elements in the several figures are illustrated for simplicity and clarity and have not been drawn to scale. The dimensions of some of the elements in the figures may be emphasized relative to other elements for facilitating an understanding of the various disclosed embodiments. In addition, common, and well-understood, elements that are useful in commercially feasible embodiments are often not depicted to provide a less obstructed view of the embodiments of the present disclosure.

### LISTING OF REFERENCE NUMERALS USED IN THE DRAWINGS

| | |
|---|---|
| snap-fit cover 102 | sheath-hub cap 800 |
| cover entrance portal 104 | sheath-hub cap hole 802 |
| cover exit portal 105 | sheath hub 804 |
| dilator snap-fit feature 106 | sheath-hub portal 805 |
| cover passageway 107 | sheath-cap snap-fit feature 806 |
| sheath-hub cap snap-fit feature 108 | dilator hub 900 |
| first medical device 201 | dilator lumen 901 |
| second medical device cap 300 | dilator shaft 902 |
| second medical device 302 | dilator ring 904 |

### DETAILED DESCRIPTION OF THE NON-LIMITING EMBODIMENT(S)

The following detailed description is merely exemplary and is not intended to limit the described embodiments or the application and uses of the described embodiments. As used, the word “exemplary” or “illustrative” means “serving as an example, instance, or illustration.” Any implementation described as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure. The scope of the disclosure is defined by the claims. For the description, the terms “upper,” “lower,” “left,” “rear,” “right,” “front,” “vertical,” “horizontal,” and derivatives thereof shall relate to the examples as oriented in the drawings. There is no intention to be bound by any expressed or implied theory in the preceding Technical Field, Background, Summary or the following detailed description. It is also to be understood that the devices and processes illustrated in the attached drawings, and described in the following specification, are exemplary embodiments (examples), aspects and/or concepts defined in the appended claims. Hence, dimensions and other physical characteristics relating to the embodiments disclosed are not to be considered as limiting, unless the claims expressly state otherwise. It is understood that the phrase “at least one” is equivalent to “a”. The aspects (examples, alterations, modifications, options, variations, embodiments and/or any equivalents thereof) are described regarding the drawings. It should be understood that the disclosure is limited to the subject matter provided by the claims, and that the disclosure is not limited to the particular aspects depicted and described. It will be appreciated that the scope of the meaning of a device configured to be coupled to an item (that is, to be connected to, to interact with the item, etc.) is to be interpreted as the device being configured to be coupled to the item, either directly or indirectly. Therefore, “configured to” may include the meaning “either directly or indirectly” unless specifically stated otherwise.

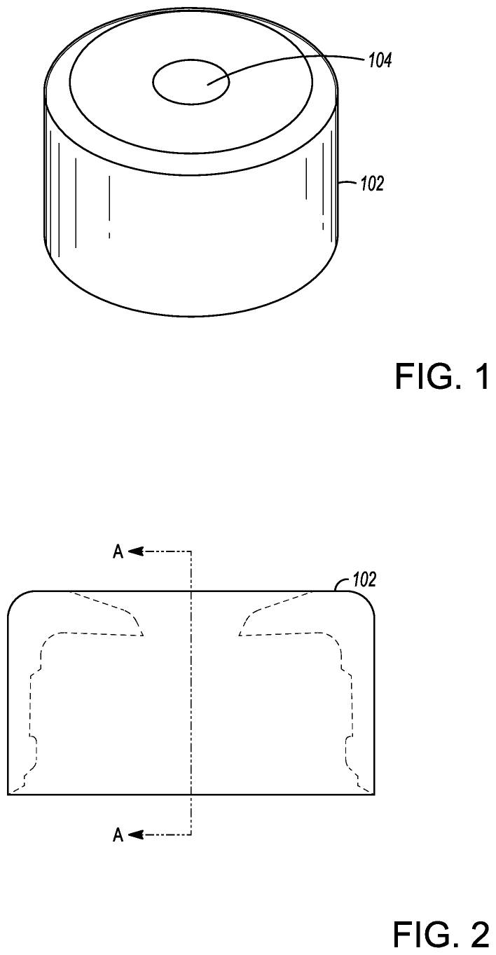

FIG. 1, FIG. 2 and FIG. 3 depict a perspective view (FIG. 1), a side view (FIG. 2) and a cross-sectional view (FIG. 3) of embodiments (implementations) of a snap-fit cover 102. FIG. 3 is a cross-sectional view taken along a cross-sectional line A-A of FIG. 2.

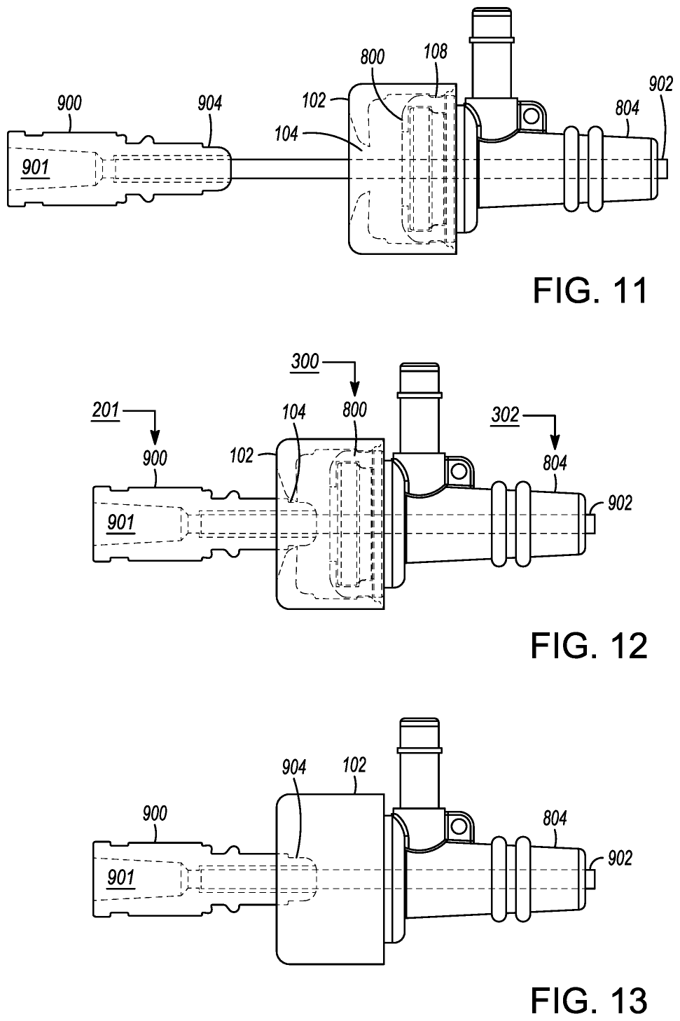

Referring to the embodiments (implementations) as depicted in FIG. 1, FIG. 2 and FIG. 3, the snap-fit cover 102 is configured to be used with (to be selectively installed to and selectively removed from) a sheath-hub cap 800 (as depicted in FIG. 12 and FIG. 13). Returning to FIG. 1, the snap-fit cover 102 is configured to be selectively installed to a sheath-hub cap 800, and the sheath-hub cap 800 is configured to be selectively installed to the sheath hub 804 (as depicted in FIG. 12 and FIG. 13). Returning to FIG. 1, the snap-fit cover 102 is also configured to be used with (to be selectively installed to and selectively removed from) a dilator hub 900 (as depicted in FIG. 11). The snap-fit cover 102 includes (defines) a cover entrance portal 104, a cover exit portal 105, and a cover passageway 107 extending between the cover entrance portal 104 and the cover exit portal 105. The cover entrance portal 104 is spaced apart from the cover exit portal 105. The cover entrance portal 104 faces the cover exit portal 105. The cover entrance portal 104 and the cover exit portal 105 are coaxially aligned along an axis that extends between the cover entrance portal 104 and the cover exit portal 105.

Referring to the embodiments (implementations) as depicted in FIG. 1, FIG. 2 and FIG. 3, the snap-fit cover 102 may have an identical stiffness to that of the dilator hub 900. The snap-fit cover 102 may be made of high-density polyethylene (HDPE) or polyethylene high-density (PEHD), which is a thermoplastic polymer produced from the monomer ethylene (sometimes called alkathene or polythene). If softer materials are desired for the snap-fit cover 102, low-density polyethylene (LDPE) may be utilized, which is a thermoplastic polymer made from the monomer ethylene; alternatively, polypropylene may be used (if desired). The material of the snap-fit cover 102 may be of comparable stiffness or softer than that of the dilator hub 900. The snap-fit cover 102 is made, preferably, of a softer material and therefore is deformable under shear stress. The inner dimension of the snap-fit cover 102 is configured to selectively fit onto the sheath-hub cap 800. The dimensions of the snap-fit cover 102 may be changed depending on the desired tightness of the fit on the sheath-hub cap 800 (as depicted in FIG. 4). The dimensions of the cover entrance portal 104 of the snap-fit cover 102 may correspond to the dimensions of the proximal portion of the dilator hub 900 (as depicted in FIG. 6). It will be appreciated that the known materials used for the dilator hub 900 and sheath-hub cap 800 are outlined below for the purpose of comparing materials.

Referring to the embodiments (implementations) as depicted in FIG. 1, FIG. 2 and FIG. 3, the snap-fit cover 102 includes (preferably) a dilator snap-fit feature 106. The dilator snap-fit feature 106 and the sheath-hub cap snap-fit feature 108 are spaced apart from each other. The dilator snap-fit feature 106 is positioned proximate to the cover entrance portal 104. The dilator snap-fit feature 106 is configured to selectively snap fit the snap-fit cover 102 to the dilator hub 900 (as depicted in FIG. 11). In accordance with another embodiment, the sheath-hub cap snap-fit feature 108 is configured to selectively snap fit the snap-fit cover 102 to the sheath-hub cap 800 configured to interface with the sheath hub 804.

Referring to the embodiments (implementations) as depicted in FIG. 1, FIG. 2 and FIG. 3, the snap-fit cover 102 includes a sheath-hub cap snap-fit feature 108. The sheath-hub cap snap-fit feature 108 is positioned proximate to the cover exit portal 105. The sheath-hub cap snap-fit feature 108 is configured to selectively snap fit the snap-fit cover 102 to the sheath-hub cap 800 (as depicted in FIG. 12).

Referring to the embodiments (implementations) as depicted in FIG. 1, FIG. 2 and FIG. 3, the dilator snap-fit feature 106 and the sheath-hub cap snap-fit feature 108 may include any feature that selectively attaches parts together, such as by pushing interlocking (interlockable) components of parts together, etc., and/or any equivalents thereof. A snap-fit feature may include a cantilever, a torsional and/or an annular feature, etc., and/or any equivalents thereof. The snap-fit feature is, preferably, an integral attachment feature with the advantages of speed and no loose parts, and/or may include non-integral attachment features (if so desired).

Referring to the embodiments (implementations) as depicted in FIG. 1, FIG. 2 and FIG. 3, the snap-fit cover 102 includes biocompatible material properties suitable for sufficient performance (such as, electric dielectric strength, thermal performance, electrical insulation, corrosion, water resistance, heat resistance) for safe performance and/or compliance with industrial and regulatory safety standards (or compatible for medical usage), etc. Reference is made to the following publication for consideration in the selection of a suitable material: Plastics in Medical Devices: Properties, Requirements, and Applications; 2nd Edition; author: Vinny R. Sastri; hardcover ISBN: 9781455732012; published: 21 Nov. 2013; publisher: Amsterdam [Pays-Bas]: Elsevier/William Andrew, [2014].

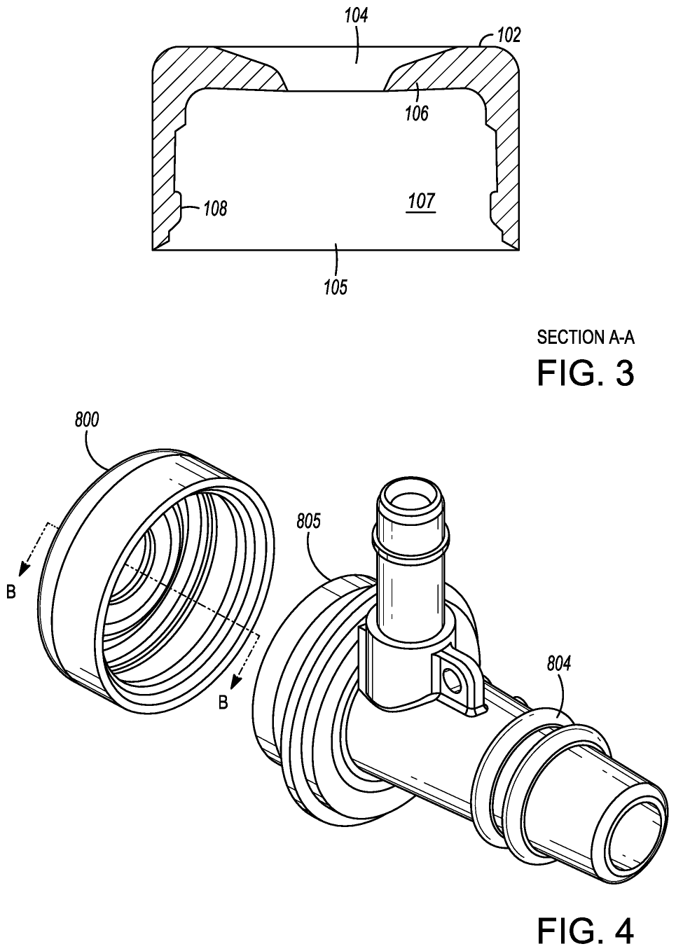

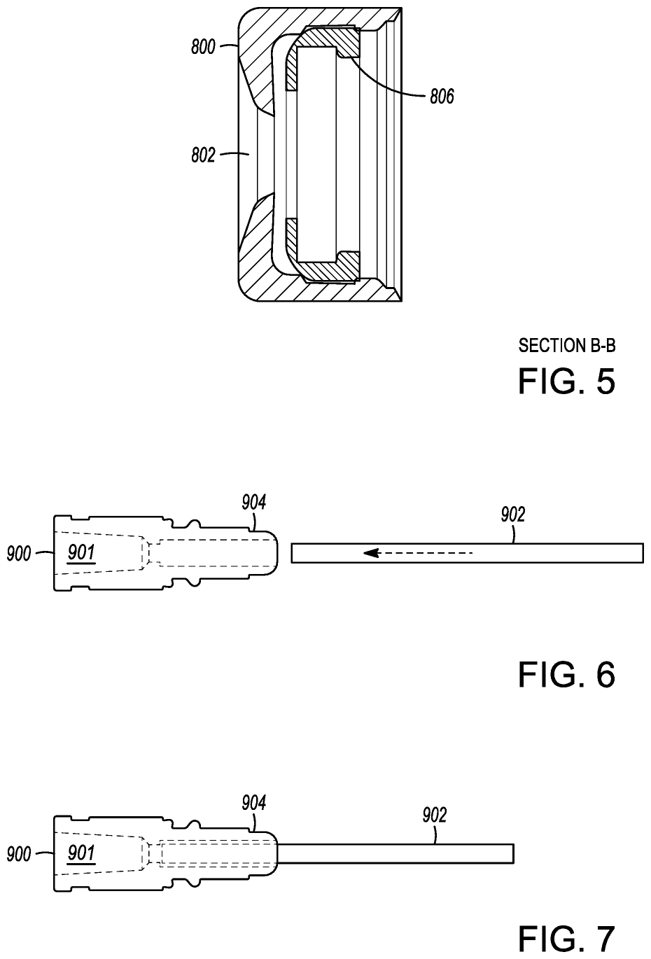

FIG. 4 and FIG. 5 depict a perspective view (FIG. 4) and a cross-sectional view (FIG. 5) of embodiments (implementations) of a sheath-hub cap 800 of a sheath hub 804 for use with the snap-fit cover 102 of FIG. 1. FIG. 5 is a cross-sectional view taken along a cross-sectional line B-B of FIG. 4.

Referring to the embodiments (implementations) as depicted in FIG. 4 and FIG. 5, the sheath-hub cap 800 includes (defines) the sheath-hub cap hole 802. The sheath-hub cap 800 includes a sheath-cap snap-fit feature 806 806 configured to selectively snap fit (connect) the sheath-hub cap 800 to the sheath hub 804. The snap-fit cover 102 of FIG. 1 is configured to snap fit over top of the sheath-hub cap 800 (as depicted in FIG. 12 and FIG. 13). The sheath hub 804 is configured to receive (at least in part) the dilator hub 900 (as depicted in FIG. 12 and FIG. 13). Returning back to FIG. 4, the sheath-hub cap 800 may be made of a polycarbonate material.

FIG. 6 and FIG. 7 depict side views of embodiments (implementations) of a dilator hub 900 for use with the snap-fit cover 102 of FIG. 1.

Referring to the embodiments (implementations) as depicted in FIG. 6 and FIG. 7, the dilator hub 900 defines a dilator lumen 901 extending along a longitudinal axis of the dilator hub 900. A dilator shaft 902 is configured to be received into the dilator lumen 901 from an end portion of the dilator hub 900. The snap-fit cover 102 (of FIG. 1) is configured to snap fit with the dilator hub 900. The dilator hub 900 may be made of high-density polyethylene (HDPE) or polyethylene high-density (PEHD), which is a thermoplastic polymer produced from the monomer ethylene (sometimes called alkathene or polythene).

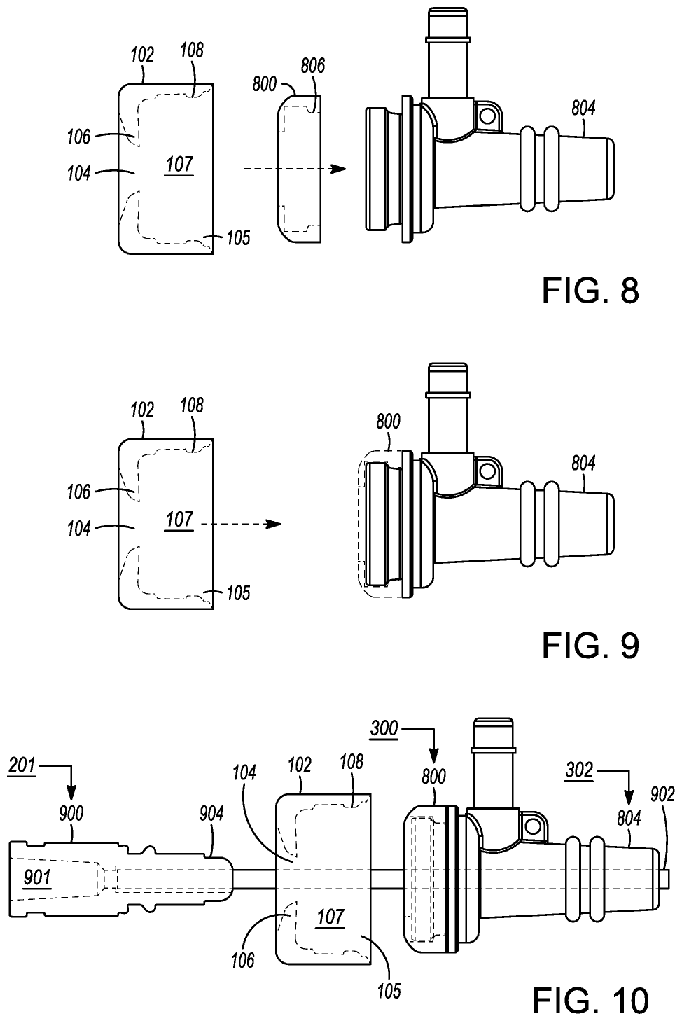

FIG. 8 to FIG. 13 depict side views of embodiments (implementations) of the snap-fit cover 102 of FIG. 1.

Referring to the embodiment (implementation) as depicted in FIG. 8, the sheath-hub cap 800 is positioned over the entrance of the sheath hub 804.

Referring to the embodiment (implementation) as depicted in FIG. 9, the sheath-cap snap-fit feature 806 (of the sheath-hub cap 800) selectively engages with (snap fits to) the entrance of the sheath hub 804 (such as, with the side wall of the sheath hub 804, etc.).

Referring to the embodiment (implementation) as depicted in FIG. 10, the dilator hub 900 is positioned proximate to the cover entrance portal 104 (of the snap-fit cover 102). It will be appreciated that the dilator shaft 902 is connected to (combined with) the dilator hub 900 before the dilator hub 900 is inserted into any of the other components; the dilator hub 900 and dilator shaft 902 are combined (or come as a combination), and then they are inserted into the sheath hub 804.

Referring to the embodiment (implementation) as depicted in FIG. 11, the dilator hub 900 is received, at least in part, into the snap-fit cover 102 (via the cover entrance portal 104). The snap-fit cover 102 is configured to selectively engage the dilator ring 904 of the dilator hub 900. It will be appreciated that the dilator ring 904 may include any type of dilator-engagement feature (and is not limited to the ring of the dilator per se). Preferably, the dilator snap-fit feature 106 is configured to selectively engage the dilator ring 904 of the dilator hub 900. As the dilator ring 904 engages with the snap-fit cover 102 and applies an amount of shear stress to the snap-fit cover 102, the opening of the snap-fit cover 102 may deform to allow entry (of the dilator hub 900) without inadvertently damaging the dilator hub 900 (especially for the case where the snap-fit cover 102 includes a relatively softer material).

Referring to the embodiment (implementation) as depicted in FIG. 11, it will be appreciated that the snap-fit cover 102 may be configured for use with any size of the dilator hub 900 and the sheath-hub cap 800, preferably with snap-fit interactions where a softer material (such as that associated with the dilator hub 900) may interact with a relatively stiffer material (such as that associated with the sheath-hub cap 800). The snap-fit cover 102 may also be extended to any device interactions where a softer material has to interact with a stiffer material to provide an intermediate material layer that interacts with the softer material to prevent unwanted damage.

Referring to the embodiment (implementation) as depicted in FIG. 11, the snap-fit cover 102 may have a comparable stiffness or may be softer than the material of the dilator hub 900 to avoid damaging the dilator hub 900. The combination of the material stiffness and the inner diameter of the circular opening (the cover entrance portal 104) of the snap-fit cover 102 may determine the locking and unlocking snap force associated with the movement of the dilator hub 900 into the sheath hub 804. The unlocking force may be as low as possible for ease of use and to prevent unintended relative motion while unlocking. The unlocking force may be sufficient to prevent the dilator hub 900 from snapping out unintentionally (away from the sheath-hub cap 800). A relatively lower force to lock the sheath-hub cap 800 and the dilator hub 900 may be desirable for ease of use.

Referring to the embodiment (implementation) as depicted in FIG. 11, in order for the snap-fit cover 102 to prevent damage to the dilator hub 900, the material stiffness may be comparable to, or softer than, the material of the dilator hub 900. This arrangement may ensure that the sheath-hub cap 800 does not damage the dilator hub 900 for the case where the dilator hub 900 snaps into the sheath-hub cap 800. The dimensions of the snap-fit cover 102 may be designed to fit tightly over the sheath-hub cap 800 so that it does not fall off during usage. The proximal hole diameter of the cover entrance portal 104 of the snap-fit cover 102 may line up with the entrance hole of the sheath-hub cap 800. The distal hole diameter of the cover entrance portal 104 may line up with the dilator hub 900 to guide the dilator hub 900 to selectively lock into the snap-fit cover 102.

Referring to the embodiment (implementation) as depicted in FIG. 12, the snap-fit cover 102 is configured to be selectively (securely) fitted to (and selectively removable from) a top section of the sheath-hub cap 800. The snap-fit cover 102 has a circular proximal opening that aligns concentrically with the opening of the sheath-hub cap 800.

Referring to the embodiments (implementations) as depicted in FIG. 11 and FIG. 12, the snap-fit cover 102 is configured to snap fit to the dilator hub 900. The snap-fit cover 102 is also configured to snap fit to the sheath-hub cap 800.

Referring to the embodiments (implementations) as depicted in FIG. 11 and FIG. 12, the snap-fit cover 102 defines the cover entrance portal 104 configured to receive the dilator hub 900. The snap-fit cover 102 is configured to snap fit to the dilator hub 900 after the cover entrance portal 104, in use, receives the dilator hub 900. The snap-fit cover 102 is also configured to snap fit to the sheath-hub cap 800 (configured to be fitted over a sheath-hub portal 805 of the sheath hub 804).

Referring to the embodiments (implementations) as depicted in FIG. 11 and FIG. 12, there is depicted a method for connecting the sheath-hub cap 800 with the dilator hub 900. The method includes snap fitting a snap-fit cover 102 to the dilator hub 900. The method also includes snap fitting the snap-fit cover 102 to the sheath-hub cap 800.

Referring to the embodiment (implementation) as depicted in FIG. 13, it will be appreciated that instead of installing the snap-fit cover 102 to the sheath-hub cap 800 (which may add an additional component), either the sheath-hub cap 800 may be modified to use a softer material or the dilator hub 900 may include a stiffer material to prevent damage to the dilator hub 900 during snapping (connection) of the dilator hub 900 into the sheath-hub cap 800.

Referring to the embodiments (implementations) as depicted in FIG. 1, FIG. 2 and FIG. 3, the sheath-hub cap 800 may be modified to use a softer material or the dilator hub 900 may use a stiffer material to prevent damage to the dilator hub 900 during snapping of (connecting) the dilator hub 900 into the sheath-hub cap 800. Alternatively, the proximal end of the dilator hub 900 may be modified to include a stiffer material including a polycarbonate snap insert at the proximal end of the dilator hub 900. For this case, the dilator hub 900 may need a stiffer polycarbonate snap insert and a softer HDPE material as part of the dilator hub 900, instead of adding an additional snap-fit cover to the sheath-hub cap 800. Similarly, a softer material may be molded directly onto the sheath-hub cap 800 instead of creating an additional component of the snap-fit cover 102 that fits on top of the sheath-hub cap 800.

Referring to the embodiment (implementation) as depicted in FIG. 10 and FIG. 12, the snap-fit cover 102 is provided for a first medical device 201 (such as the dilator hub 900) and a second medical device cap 300 (such as the sheath-hub cap 800) configured to be selectively installed to a second medical device 302 (such as the sheath hub 804). The snap-fit cover 102 is configured to snap fit to the first medical device 201 (such as, the dilator hub 900). The snap-fit cover 102 also being configured to snap fit to the second medical device cap 300 (such as the sheath-hub cap 800) of the second medical device 302 (such as the sheath hub 804).

The following is offered as further description of the embodiments, in which any one or more of any technical feature (described in the detailed description, the summary and the claims) may be combinable with any other one or more of any technical feature (described in the detailed description, the summary and the claims). It is understood that each claim in the claims section is an open ended claim unless stated otherwise. Unless otherwise specified, relational terms used in these specifications should be construed to include certain tolerances that the person skilled in the art would recognize as providing equivalent functionality. By way of example, the term perpendicular is not necessarily limited to 90.0 degrees, and may include a variation thereof that the person skilled in the art would recognize as providing equivalent functionality for the purposes described for the relevant member or element. Terms such as “about” and “substantially”, in the context of configuration, relate generally to disposition, location, or configuration that are either exact or sufficiently close to the location, disposition, or configuration of the relevant element to preserve operability of the element within the disclosure which does not materially modify the disclosure. Similarly, unless specifically made clear from its context, numerical values should be construed to include certain tolerances that the person skilled in the art would recognize as having negligible importance as they do not materially change the operability of the disclosure. It will be appreciated that the description and/or drawings identify and describe embodiments of the apparatus (either explicitly or inherently). The apparatus may include any suitable combination and/or permutation of the technical features as identified in the detailed description, as may be required and/or desired to suit a particular technical purpose and/or technical function. It will be appreciated that, where possible and suitable, any one or more of the technical features of the apparatus may be combined with any other one or more of the technical features of the apparatus (in any combination and/or permutation). It will be appreciated that persons skilled in the art would know that the technical features of each embodiment may be deployed (where possible) in other embodiments even if not expressly stated as such above. It will be appreciated that persons skilled in the art would know that other options may be possible for the configuration of the components of the apparatus to adjust to manufacturing requirements and still remain within the scope as described in at least one or more of the claims. This written description provides embodiments, including the best mode, and also enables the person skilled in the art to make and use the embodiments. The patentable scope may be defined by the claims. The written description and/or drawings may help to understand the scope of the claims. It is believed that all the crucial aspects of the disclosed subject matter have been provided in this document. It is understood, for this document, that the word “includes” is equivalent to the word “comprising” in that both words are used to signify an open-ended listing of assemblies, components, parts, etc. The term “comprising”, which is synonymous with the terms “including,” “containing,” or “characterized by,” is inclusive or open-ended and does not exclude additional, unrecited elements or method steps. Comprising (comprised of) is an “open” phrase and allows coverage of technologies that employ additional, unrecited elements. When used in a claim, the word “comprising” is the transitory verb (transitional term) that separates the preamble of the claim from the technical features of the disclosure. The foregoing has outlined the non-limiting embodiments (examples). The description is made for particular non-limiting embodiments (examples). It is understood that the non-limiting embodiments are merely illustrative as examples.

1. An apparatus for use with a first medical device and a second medical device cap configured to be selectively installed to a second medical device, the apparatus, comprising:
a snap-fit cover configured to snap fit to the first medical device; and
the snap-fit cover also being configured to snap fit to the second medical device cap of the second medical device.

2. The apparatus of claim 1, wherein:
the first medical device is a dilator hub and the second medical device is a sheath-hub.

3. An apparatus for use with a dilator hub and a sheath-hub cap configured to be selectively installed to a sheath hub having a sheath-hub portal, the apparatus, comprising:
a snap-fit cover defining a cover entrance portal configured to receive, at least in part, the dilator hub; and
the snap-fit cover being configured to snap fit to a portion of the dilator hub; and
the snap-fit cover also being configured to snap fit to the sheath-hub cap configured to be selectively installed after the sheath-hub cap has been installed to the sheath-hub portal of the sheath hub.

4. The apparatus of claim 3, wherein:
the snap-fit cover includes:
a cover exit portal spaced apart from the cover entrance portal; and
a cover passageway extending between the cover entrance portal and the cover exit portal; and
the cover entrance portal is spaced apart from the cover exit portal; and
the cover entrance portal faces the cover exit portal; and
the cover entrance portal and the cover exit portal are coaxially aligned along an axis that extends between the cover entrance portal and the cover exit portal.

5. The apparatus of claim 3, wherein:
the snap-fit cover has an identical stiffness to the dilator hub.

6. The apparatus of claim 3, wherein:
the snap-fit cover is softer than the dilator hub.

7. The apparatus of claim 3, wherein:
the snap-fit cover includes:
a dilator snap-fit feature; and
the dilator snap-fit feature is positioned proximate to the cover entrance portal; and
the dilator snap-fit feature is configured to selectively snap fit the snap-fit cover to the dilator hub.

8. The apparatus of claim 3, wherein:
the snap-fit cover includes:
a sheath-hub cap snap-fit feature being positioned proximate to a cover exit portal; and
the sheath-hub cap snap-fit feature being configured to selectively snap fit the snap-fit cover to the sheath-hub cap.

9. The apparatus of claim 3, wherein:
the snap-fit cover is configured to be selectively installed to the sheath-hub cap; and
the snap-fit cover includes:
a sheath-hub cap snap-fit feature being positioned proximate to a cover exit portal; and
the sheath-hub cap snap-fit feature also being configured to selectively snap fit the snap-fit cover to the sheath-hub cap configured to interface with the sheath hub.

10. The apparatus of claim 3, wherein:
the dilator hub is received, at least in part, into the snap-fit cover via the cover entrance portal.

11. The apparatus of claim 3, wherein:
the snap-fit cover is configured to selectively engage a dilator ring of the dilator hub.

12. The apparatus of claim 3, wherein:
the snap-fit cover includes a dilator snap-fit feature configured to selectively engage a dilator ring of the dilator hub.

13. The apparatus of claim 3, wherein:
the snap-fit cover is configured to be selectively installed to the sheath-hub cap; and
the snap-fit cover is also configured to be selectively fitted to a top section of the sheath-hub cap.

14. A method for connecting a sheath-hub cap of a sheath hub with a dilator hub, the method comprising:
snap fitting a snap-fit cover to the dilator hub; and
snap fitting the snap-fit cover to the sheath-hub cap.

15. The method of claim 14, further comprising:
positioning the sheath-hub cap over an entrance of the sheath hub; and
snap fitting the snap-fit cover to sheath-hub cap.

16. The method of claim 15, further comprising:
selectively engaging a sheath-cap snap-fit feature of the sheath-hub cap with the entrance of the sheath hub.

17. The method of claim 14, further comprising:
positioning the dilator hub, in use, proximate to the cover entrance portal of the snap-fit cover.

18. The method of claim 14, further comprising:
receiving, at least in part, the dilator hub into the snap-fit cover via an cover entrance portal.

19. The method of claim 14, further comprising:
selectively engaging the snap-fit cover with a dilator ring of the dilator hub.

20. The method of claim 19, further comprising:
selectively engaging a dilator snap-fit feature of the snap-fit cover with the dilator ring of the dilator hub.
